(19) United States
(12) Patent Application Publication
(10) Pub. No.: US 2010/0108778 A1
(43) Pub. Date: May 6, 2010

(54) DEVICE FOR CONTAINING AND RELEASING A VOLATILE SUBSTANCE

(76) Inventor: Steven J. Greenland, Hampton, NH (US)

Correspondence Address: JONES DAY, 222 EAST 41ST ST, NEW YORK, NY, 10017, US

(21) Appl. No.: 12/262,082

(22) Filed: Oct. 30, 2008

(51) Int. Cl. A61L 9/12; B65B 3/02; B65B 7/28

(52) U.S. Cl. 239/56; 239/53; 53/412; 53/456; 53/471

(57) ABSTRACT

A device for containing and releasing a volatile substance having a lower vapor barrier layer; a fluid reservoir melt bonded to the lower vapor barrier layer and a volatile substance absorbed in the fluid reservoir; and an upper vapor barrier layer peelably sealed to the lower vapor barrier layer to form a fluid tight cavity containing the fluid reservoir. The volatile substance can be a fragrance. The fluid reservoir includes a composite material formed from a fibrous component and a heat sealable thermoplastic component. Methods of making the device are also provided.

### FIELD OF THE INVENTION

This invention generally relates to a device for containing and releasing a volatile substance such as a fragrance. The device includes a fluid reservoir having the volatile substance absorbed therein. The invention is also directed to a method for making such device.

### BACKGROUND OF THE INVENTION

Various types of devices or dispensers have been made for delivering a fragrance or other volatile substance to a targeted area. One example is a static type delivery device in which a volatile substance is delivered to the environment by direct interface with the air. These devices use various structures and materials for containing and facilitating release of the volatile substance. Examples of such structures include an air freshener card that is suspended from a string or wire, a colloidal gel, an absorbent matrix material anchored in an injection molded container, or a micropermeable film with a perforated cover through which the volatile substance is released.

In these prior static type delivery devices, natural fibers are often used to make a fluid reservoir for containing the volatile substance. Such fluid reservoirs are often attached to the device. But chemicals commonly present in a fragrance or other volatile substance react with the majority of available bonding agents that could be used to attach the reservoir to the device, in addition to simultaneously acting on the natural fibers of the fluid reservoir.

Previous static type devices using a paper reservoir were often required to use a separate top layer of material that surrounds the paper reservoir in order to attach the paper to the device because it would be very difficult to otherwise attach the paper to the device. However, this makes the device more costly to manufacture and negatively impacts the performance of the device.

In some other devices, a plastic is used as the reservoir for carrying the fragrance. But a plastic reservoir does not effectively release the fragrance as plastic lacks adequate surface area and is not sufficiently oleophobic.

Thus, due to these and other limitations of the previous devices, there is a need for a static type delivery device that has a sufficient surface area to contain and release a volatile substance, includes a fluid reservoir that is adequately bonded to the device, and can resist degradation by the volatile chemicals contained in the fluid reservoir. Also, there is a need for a cost-effective and efficient method of manufacturing such device.

### SUMMARY OF THE INVENTION

The present invention is directed to an improved static type delivery device which includes a fluid reservoir which both binds and efficiently releases a volatile substance such as a fragrance to a target area. The fluid reservoir is adhered to a lower vapor barrier layer and contained within a cavity formed by the lower vapor barrier layer and an upper vapor barrier layer that are peelably sealed together. Such device can be manufactured in a cost-effective and efficient manner.

One embodiment is directed to a device for containing and releasing a volatile substance. The device comprises a lower vapor barrier layer having a first surface and a second surface, wherein the second surface comprises a heat sealable thermoplastic material. The device also includes a fluid reservoir comprising a composite material. The composite material includes a fibrous component and a heat sealable thermoplastic component. A heat seal is formed between the fluid reservoir and the second surface of the lower vapor barrier layer, such that the fluid reservoir and the second surface of the lower vapor barrier layer are in a mechanical thermoplastic melt bonded relationship. Also, a volatile substance is absorbed into the fluid reservoir and an upper vapor barrier layer is disposed directly over the fluid reservoir. A peel seal attaches the second surface of the lower vapor barrier layer to the upper vapor barrier layer, thereby forming a fluid tight cavity containing the fluid reservoir. Upon separation of the upper vapor barrier layer and the lower vapor barrier layer, the fluid reservoir remains adhered to the lower vapor barrier layer and the fluid reservoir is exposed to the atmosphere and capable of releasing the volatile substance.

In this embodiment, the first surface of the lower vapor barrier layer may comprise a pressure sensitive adhesive. Also, the first surface of the lower vapor barrier layer may comprise a repositionable adhesive. The fluid reservoir may be an uncoated paper at least partially treated with a thermoplastic polyolefin dispersion. In a certain embodiment, the heat sealable thermoplastic component is formed from a thermoplastic aqueous dispersion. The thermoplastic component may comprise an aqueous dispersion of ethylene acrylic acid copolymer. At least one of the lower vapor barrier layer and the upper vapor barrier layer may comprise a polyethylene based material, a heat sealable laminate material, an aluminum foil, a layer of oriented polyester, or a combination thereof. Also, the fibrous component may include cellulose fiber, cotton fiber, or an organic resin fiber. In a certain embodiment, the volatile substance is a fragrance which may be an oil. The device may be planar. In another embodiment, the device further comprises a release liner adhered to the first surface of the lower vapor barrier layer. The device may have a carrier adhered to the first surface of the lower vapor barrier layer.

Another embodiment is a device for containing and releasing a fragrance that comprises a lower vapor barrier layer having a first surface and a second surface, wherein the first surface comprises a pressure sensitive adhesive and the second surface comprises a heat sealable thermoplastic material. This device further includes a fluid reservoir comprising an uncoated paper stock at least partially treated with a thermoplastic polyolefin dispersion. A fragrance is absorbed into the fluid reservoir. The device also comprises an upper vapor barrier layer disposed directly on the fluid reservoir, wherein at least one of the lower vapor barrier layer and the upper vapor barrier layer comprises a polyethylene based material. In this embodiment, a peel seal attaches the lower vapor barrier layer to the upper vapor barrier layer, thereby forming a fluid tight cavity containing the fluid reservoir. Upon separation of the upper vapor barrier layer and the lower vapor barrier layer, the fluid reservoir remains adhered to the lower vapor barrier layer and the fluid reservoir is exposed to the atmosphere and capable of releasing the fragrance.

Yet another embodiment is a method of making a device for containing and releasing a volatile substance. The method includes the step of providing a lower vapor barrier layer having a first surface and a second surface, wherein the second surface comprises a heat sealable thermoplastic material, and disposing a pressure sensitive adhesive on the first surface of the lower vapor barrier layer. The method further comprises forming a fluid reservoir comprising a composite material comprising a fibrous component and a heat sealable thermoplastic component, and applying a heat seal between the fluid reservoir and the second surface of the lower vapor barrier layer to form a mechanical thermoplastic melt bond between the fluid reservoir and the second surface of the lower vapor barrier layer. The method further comprises absorbing a volatile substance into the fluid reservoir and disposing an upper vapor barrier layer directly on the fluid reservoir. A peel seal is applied between the lower vapor barrier layer and the upper vapor barrier layer, thereby forming a fluid tight cavity containing the fluid reservoir. Upon separation of the upper vapor barrier layer and the lower vapor barrier layer, the fluid reservoir remains adhered to the lower vapor barrier layer and the fluid reservoir is exposed to the atmosphere and capable of releasing the volatile substance.

### BRIEF DESCRIPTION OF THE FIGURES

Reference is made to a brief description of the drawings, which are intended to illustrate a certain embodiment of the device described herein, wherein similar reference characters denote similar elements in the different views.

FIG. 1 illustrates an embodiment of a device for delivering a volatile substance which has been partially peeled apart; and

FIG. 2 is a cross sectional view of the device of FIG. 1 taken along line 2-2 of FIG. 1.

### DETAILED DESCRIPTION

The device for containing and releasing a volatile substance comprises three structural layers, i.e., a lower barrier layer that may be adhered to a host surface; a fluid reservoir bonded to the lower barrier layer and capable of holding and releasing a volatile substance; and an at least partially removable upper barrier layer which is peelably sealed, preferably hermetically, to the lower barrier layer. A volatile substance is absorbed into the fluid reservoir. Preferably, the lower barrier layer is a lower vapor barrier layer and the upper barrier layer is an upper vapor barrier layer.

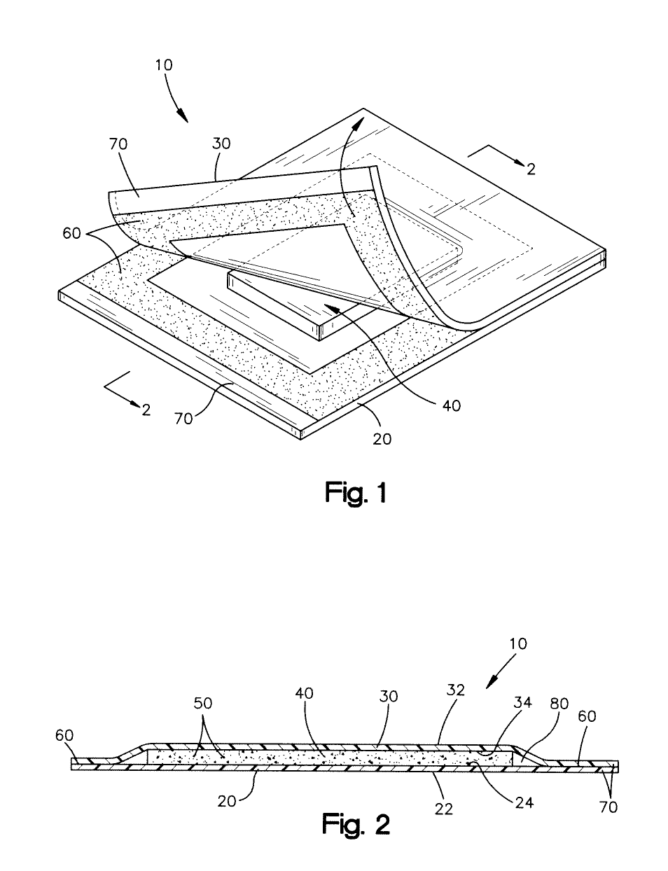

FIGS. 1 and 2 show an embodiment of the device 10 for containing and releasing a volatile substance. As shown in FIGS. 1 and 2, the device 10 comprises a lower barrier layer 20, a fluid reservoir 40 that absorbs a volatile substance 50 disposed on the lower barrier layer 20, an upper barrier layer 30 disposed directly on the fluid reservoir 40, and a peel seal 60 joining the lower and upper barrier layers 20 and 30, thus forming a sealed fluid tight compartment or cavity 80 (shown in FIG. 2) for containing the fluid reservoir 40 and volatile substance 50.

As shown in FIG. 2, the lower barrier layer 20 of the device 10 has a first surface 22 and a second surface 24, and the upper barrier layer 30 has a first surface 32 and a second surface 34. The lower barrier layer 20 and upper barrier layer 30 may comprise any suitable materials. which may be the same or different in each layer. Also, the lower and upper barrier layers 20 and 30 may each comprise one or more layers of materials. The first surface and second surface of the lower and upper barrier layers 20 and 30 may comprise different or the same materials. For example, the first surface 22 of the lower barrier layer 20, which is further away from the fluid reservoir 40, preferably comprises a pressure sensitive adhesive. The second surface 24 of the lower barrier layer 20, which is adjacent to and bonded to the fluid reservoir 40, preferably comprises a heat sealable thermoplastic material.

At least one of the lower and upper barrier layers 20 and 30 is constructed of a vapor barrier material. Preferably, both layers are constructed of a vapor barrier material. For example, the lower and upper barrier layers 20 and 30 may be constructed with biaxially oriented polyester film, polypropylene film, high density polyethylene film, “Barex” acrylonitrile co-polymer film, cast PET or PETG film, aluminum foil, PVDC film, co-extruded films containing EVOH, PVA film, polyamide film, vinyl film, composite laminations or coatings containing the same, or any other suitable materials known in the art. Preferred materials for the lower barrier layer 20 and upper barrier layer 30 include a polyethylene based material, a layer of oriented polyester, an aluminum foil, and a heat sealable laminate material. The material for the lower and upper barrier layers 20 and 30 is preferably a heat sealable, flexible, foil bearing plastic vapor barrier laminate.

In one embodiment, at least one of the lower and upper barrier layers 20 and 30 is constructed with a peelable olefin sealant layer which is commercially available from Amcor Flexible Packaging and sold under the trade name “Rayopeel”.

Alternatively, one or both of the lower and upper barrier layers 20 and 30 is constructed with a lamination of polyethylene terephthalate (PET)/foil and polyethylene blend material. The preferred lower barrier layer 20 is a lamination of oriented PET/foil and linear low density polyethylene (LLDPE). In a certain embodiment, the upper barrier layer 30 includes polyethylene blend material. The polyethylene blend material of the upper barrier layer 30 is heat sealable to the LLDPE layer of the lower barrier layer 20 and provides a peelable cohesive rupture peel seal. The preferred polyethylene blend layer is Rayopeel.

In a preferred embodiment the materials included in the lower barrier layer 20 or the upper barrier layer 30, from the outermost layer (i.e., the layer furthest from the fluid reservoir 40) inward, are as follows: 48 gage biaxially oriented polyester film/adhesive/0.0030 inch aluminum foil/adhesive/40-50 microns “Rayopeel-R” co-extruded olefin film. The lower barrier layer 20 and the upper barrier layer 30 may both comprise this structure.

In another embodiment, the lower barrier layer 20 is formed from a semi-rigid, foil bearing laminate structure. Such a laminate structure from outermost layer inward is a follows: 9 point SBS bleach board/7 lb. per ream polyethylene extrusion tie layer/0.0030 inch aluminum foil/primer/22 pounds per ream polyethylene extrusion coating. The lower and upper barrier layers 20 and 30 may be of any desired size and shape. The lower and upper barrier layers 20 and 30 may have the same or different sizes and shapes. The upper barrier layer 30 preferably corresponds in shape and size to the lower barrier layer 20 as shown in FIGS. 1 and 2.

The device 10 may further include a release liner (not shown) attached to the first surface 22 of the lower barrier layer 20. In such case, the lower barrier layer 20 preferably includes an adhesive, such as a pressure sensitive adhesive, and the release liner is attached to the pressure sensitive adhesive. For example, the lower barrier layer 20 is coated with a pressure sensitive adhesive on its first surface 22, which is further covered by the release liner, such as a siliconized release liner. Once the release liner is removed, a user may attach or otherwise mount the device 10 to any desired carrier or target.

As shown in FIGS. 1 and 2, the fluid reservoir 40 is attached to the second surface 24 of the lower barrier layer 20. The materials suitable for making the fluid reservoir 40 must be chemically inert to the volatile substance 50, and also be able to absorb and hold enough volatile substance 50 to provide a desired release and sustained delivery of the volatile substance 50.

In a certain embodiment, the fluid reservoir 40 comprises a composite material comprising a fibrous component and a thermoplastic heat sealable component. The fibrous component may be of any shape, preferably planar. Also, the fibrous component of the fluid reservoir 40 may be formed from woven or non-woven fiber webs of polyester, rayon, cellulose, cotton, polyethylene, nylon, composites of the above., or any other suitable materials. In one embodiment, the fibrous component is an organic resin fiber. In another embodiment, the fibrous component of the fluid reservoir 40 is constructed with a non-woven polyester fiber web with a basis weight in the range of 35 to 100 grams per square yard. In a preferred embodiment, the fibrous component is a cellulose fiber.

Preferably, the fibrous component comprises uncoated paper stock. Paper stock or fiber, either alone or blended with other natural fiber such as cotton, has been found to be able to hold and release the volatile substance in a controlled manner. Though numerous paper grades can be used to make the fibrous component of the fluid reservoir, uncoated paper is preferred due to the microscopic texturing of its surface, which enables it to release the volatile substance within the reservoir to the surrounding atmosphere more efficiently. Preferably, the uncoated paper has a classified grade of cover stock, card or tag. A particularly preferred uncoated paper stock is commonly sold as 110 pound index card, which has a thickness between 10.5 to 11.0 mils (10⁻³ inch). These uncoated paper grades are capable of partially binding, holding and releasing the volatile substance in a controlled manner, and are readily available and economical.

Paper and natural fibers of the fluid reservoir 40 can gradually absorb typical volatile fragrances, which are often blended with plasticizing agents or extraction solvents that react with the paper and natural fibers. Advantageously, the paper reservoir can bind the volatile substance, yet release the volatile substance over time.

To form a bond between the fluid reservoir 40 and the lower barrier layer 20, the fibrous component of the fluid reservoir 40 may be topically sized or treated with a thermoplastic heat sealable component compatible with the lower barrier layer 20 to create a mechanical tie layer.

The thermoplastic heat sealable component is preferably formed from a dispersion such as a thermoplastic aqueous dispersion. A suitable viscosity of the thermoplastic dispersion is less than 500 centipoise. At this viscosity the thermoplastic dispersion readily wets the paper or fibrous component. The thermoplastic aqueous dispersion is preferably a thermoplastic polyolefin dispersion. In one embodiment, the thermoplastic aqueous dispersion includes ethylene acrylic acid copolymer. Thermoplastic dispersions of ethylene acrylic acid copolymer can be purchased from Paramelt B. V. of Heerhugawaard, the Netherlands, or from Michelman Inc. of Cincinnati, Ohio, sold under the trade name “Michem Prime 2960”. Also, ethylene acrylic acid copolymer resin can be purchased from Dow Chemical Company under the trade name “Primacor”. This resin may be transformed into a thermoplastic dispersion by mechanically agitating and heating the resin in an alkaline solution. The resulting thermoplastic dispersion, a fine particle-dispersed solution, can be readily applied to the fibrous component of the fluid reservoir. Such dispersion can wet into and around the fibrous component of the fluid reservoir, which allows the fluid reservoir to adhere to the lower barrier layer. Other variations of the thermoplastic dispersions may also be used to treat the fibrous component of the fluid reservoir.

Any method of coating can be used to apply the thermoplastic dispersion (or other heat sealable thermoplastic component) to the fibrous component. For example, the thermoplastic dispersion may be applied by spraying, gravure, rod, roll coating, or reverse rolling. Generally, the thermoplastic dispersion permeates the surface of the fibrous component such that an effective bond can be formed between the second surface of the lower surface layer and the coated surface of the fibrous component, though the thermoplastic dispersion is not absorbed throughout the fibrous component.

As an alternative to a coating method, the fibrous component may be impregnated with the thermoplastic dispersion, such as by incorporating the thermoplastic dispersion into the fibrous component or paper during manufacture, to achieve a uniform distribution of the thermoplastic dispersion throughout the fibrous component. Another alternative to coating is infusing the fibrous component with dry thermoplastic polymer particles to form a uniform distribution of the thermoplastic particles throughout the fibrous component of the fluid reservoir.

Preferably, the thermoplastic dispersion is applied using a coating method.

After the heat sealable thermoplastic component has been applied to the fibrous component to form the composite material of the fluid reservoir, the fluid reservoir is dried (for example, to remove water from the heat sealable thermoplastic component) by any suitable technique. Preferably, the fluid reservoir is dried by evaporation and the application of heat. Thus, when dried and later heated to thermal activation temperature, the fibrous component and the heat sealable thermoplastic component form a mechanical and chemical bond with each other.

The fluid reservoir 40 comprising the fibrous thermoplastic heat sealable composite material is then adhered to the lower barrier layer 20. Preferably, the fluid reservoir 40 is adhered to the lower barrier layer 20 by a heat seal such that the fluid reservoir 40 and the lower barrier layer 20 are in a mechanical thermoplastic melt bonded relationship. To achieve this bonding, the second surface 22 of the lower barrier layer 20 preferably comprises a heat sealable thermoplastic material. The lower barrier layer is preferably a lamination of oriented PET/foil and LLDPE. The LLDPE layer of the lower barrier layer and the surface of the fluid reservoir can be heat sealed and bonded together through the use of a patterned heat seal die as known to one skilled in the art. In particular, to adhere the fluid reservoir to the lower barrier layer, the fluid reservoir is disposed on the second surface of the lower barrier layer, and the fluid reservoir and lower barrier layer are together placed between a patterned heat seal die and a resilient backing plate. Preferably, the first surface of the lower barrier layer is adjacent to the patterned heat seal die, and the surface of the fluid reservoir that is not adhered to the lower barrier layer is adjacent to the resilient backing plate. When the fluid reservoir has been formed by coating a surface of the fibrous component with the heat sealable thermoplastic component, the coated surface of the fibrous component is placed adjacent to the second surface of the lower barrier layer before applying the heat seal.

After the heat seal has been applied, the fluid reservoir is allowed to cool, preferably to room temperature. After the fluid reservoir has sufficiently cooled, the fragrance or other volatile substance is applied to the fluid reservoir so that it becomes absorbed into the fluid reservoir. The fluid reservoir 40 confines the volatile substance 50, thus preventing the volatile substance 50 from diffusing into the peel seal 60 during manufacturing, and, when the device 10 is peeled open, loosely binds the volatile substance and prevents its spillage, particularly when a low viscosity volatile substance, e.g., fragrance, is used.

The volatile substance may be applied by any method as known to one skilled in the art. For example, the volatile substance may be applied to the fluid reservoir using a micropipetting operation or by a coating method such as, but not limited to, spraying, gravure, rod, rollcoat, and reverse rollcoat. Complete saturation and absorbent uptake of the volatile substance by the fluid reservoir 40 is not required immediately but may be achieved over time.

A preferred concentration of the volatile substance in the fluid reservoir is 10 to 25 microliters of volatile substance per 100 microliters of reservoir.

As used herein, the term “volatile substance” refers to a substance, often a liquid, that evaporates readily at room temperature and preferably gives off an odor upon evaporation that is suitable to condition, modify or otherwise change the ambient atmosphere. Examples of the volatile substance include, but are not limited to, a fragrance, an air freshener, a deodorizer, an insecticide, an insect repellant, or a volatile corrosion inhibitor that is used as a preservative system. In a preferred embodiment, the volatile substance is a fragrance. A fragrance is a low to medium viscosity liquid, and may comprise an oil.

Subsequent to at least partially absorbing the volatile substance 50 into the fluid reservoir 40, the fluid reservoir 40 is covered with the upper barrier layer 30. The lower barrier layer 20 is attached to the upper barrier layer 30 to form a fluid tight cavity 80 that contains the fluid reservoir 40. The second surface 34 of the upper barrier layer 30 and the second surface 24 of the lower barrier layer 20 are preferably joined together at the perimeter by a peel seal 60.

The cavity 80 must be substantially fluid tight. The cavity 80 may contain any amount of the volatile substance 50. For example, for a small planar device 10, cavity 80 contains 25 milligrams to about 150 milligrams of volatile substance per square inch within cavity 80.

Peel seal 60 may be formed by any appropriate method. For example, peel seal 60 may comprise a heat seal (through the use of a patterned heat seal die), a melt bond, another appropriate adhesive, or a cohesively rupturing thermoplastic film. In a preferred embodiment, the peel seal 60 is a hermetic bond between the second surface 24 of the lower barrier layer 20 and the second surface 34 of the upper barrier layer 30 that is formed by external application of heat and pressure.

The upper barrier layer 30 may be partially unpeeled or completely unpeeled and removed from the lower barrier layer 20 prior to use. In other embodiments, the peel seal 60 may be resealable, thereby allowing a user to reclose the sampler device 10 and store it for later use.

In a certain embodiment as shown in FIGS. 1 and 2, the upper and lower barrier layers 30 and 20 also extend beyond the area of cavity 80 and peel seal 60 to provide a tab 70 to enable the user to initiate peeling of the upper barrier layer 30 from the lower barrier layer 20. Thus, a scissors or a tear notch are not required to open the sampler device 10, though may be used if desired.

When the device 10 is peeled open, the fluid reservoir 40 and the volatile substance 50 absorbed therein become exposed to the atmosphere. The fluid reservoir 40 remains adhered to the lower barrier layer 20 and releases the volatile substance 50 into the surrounding atmosphere.

The device 10 for containing and releasing a volatile substance may be disposable, or for a single use. For example, the device 10 can be used as a disposable dispenser for the short term delivery of a fragrance or volatile organic chemical. The device 10 for a single use may be discarded once the volatile substance has evaporated.

The device 10 for containing and releasing a volatile substance may be of any shape. In a preferred embodiment, the device 10 is planar. For example, the device 10 may be an air static volatile delivery device in the form of a pressure sensitive adhesive label.

A user may attach or mount the device 10 to any desired carrier or target. Such carrier or target includes, without limitation, a magazine, advertisement, wall, basement, bathroom, closet, kitchen or other room, trash receptacle, clothing or other fabric, car interior, picnic table, or vacuum cleaner.

In a preferred embodiment, a method of making the device 10 described above comprises providing a lower barrier layer, having a first surface and a second surface, wherein the second surface comprises a heat sealable thermoplastic material, and disposing a pressure sensitive adhesive on the first surface of the lower barrier layer. This method also includes forming a fluid reservoir comprising a composite material comprising a fibrous component and a thermoplastic heat sealable component, applying a heat seal between the fluid reservoir and the second surface of the lower barrier layer to form a mechanical thermoplastic melt bond, and absorbing a volatile substance into the fluid reservoir. The method further includes disposing an upper barrier layer directly on the fluid reservoir, and applying a peel seal between the lower barrier layer and the upper barrier layer, thereby forming a fluid tight cavity containing the fluid reservoir. Upon separation of the upper barrier layer and the lower barrier layer, the fluid reservoir remains adhered to the lower barrier layer and the fluid reservoir is exposed to the atmosphere and capable of releasing the volatile substance.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

I claim:

1. A device for containing and releasing a volatile substance comprising:
a lower vapor barrier layer having a first surface and a second surface, wherein the second surface comprises a heat sealable thermoplastic material;
a fluid reservoir comprising a composite material comprising a fibrous component and a heat sealable thermoplastic component;
a heat seal between the reservoir and the second surface of the lower vapor barrier layer, such that the fluid reservoir and the second surface of the lower vapor barrier layer are in a mechanical thermoplastic melt bonded relationship;
a volatile substance absorbed into the fluid reservoir;
an upper vapor barrier layer disposed directly or the fluid reservoir; and
a peel seal attaching the second surface of the lower vapor barrier layer to the upper vapor barrier layer, thereby forming a fluid tight cavity containing the fluid reservoir,
wherein upon separation of the upper vapor barrier layer and the lower vapor barrier layer, the fluid reservoir remains adhered to the lower vapor barrier layer, and the fluid reservoir is exposed to the atmosphere and capable of releasing the volatile substance.

2. The device of claim 1, wherein the first surface of the lower vapor barrier layer comprises a pressure sensitive adhesive.

3. The device of claim 1, wherein the first surface of the lower vapor barrier layer comprises a repositionable adhesive.

4. The device of claim 1, wherein the fluid reservoir is an uncoated paper at least partially treated with a thermoplastic polyolefin dispersion.

5. The device of claim 1, wherein the heat sealable thermoplastic component is formed from a thermoplastic aqueous dispersion.

6. The device of claim 1, wherein the heat sealable thermoplastic component comprises an aqueous dispersion of ethylene acrylic acid copolymer.

7. The device of claim 1, wherein at least one of the lower vapor barrier layer and the upper vapor barrier layer comprises a polyethylene based material.

8. The device of claim 1I wherein at least one of the lower vapor barrier layer and the upper vapor barrier layer comprises a heat sealable laminate material.

9. The device of claim 1, wherein at least one of the lower vapor barrier layer and the upper vapor barrier layer includes an aluminum foil.

10. The device of claim 1, wherein at least one of the lower vapor barrier layer and the upper vapor barrier layer includes a layer of oriented polyester.

11. The device of claim 1, wherein the fibrous component comprises cellulose fiber.

12. The device of claim 1, wherein the fibrous component comprises cotton fiber.

13. The sampler device of claim 1, wherein the fibrous component comprises an organic resin fiber.

14. The device of claim 1, wherein the volatile substance is a fragrance.

15. The device of claim 14, wherein the fragrance comprises an oil.

16. The device of claim 1, wherein the device is planar.

17. The device of claim 1, further comprising a release liner adhered to the first surface of the lower vapor barrier layer.

18. The device of claim 1, further comprising a carrier adhered to the first surface of the lower vapor barrier layer.

19. A device for containing and releasing a fragrance comprising:
a lower vapor barrier layer having a first surface and a second surface, wherein the first surface comprises a pressure sensitive adhesive and the second surface comprises a heat sealable thermoplastic material;
a fluid reservoir comprising a composite material comprising an uncoated paper stock at least partially treated with a thermoplastic polyolefin dispersion;
a fragrance absorbed into the fluid reservoir;
an upper vapor barrier layer disposed directly on the fluid reservoir, wherein at least one of the lower vapor barrier layer and the upper vapor barrier layer comprises a polyethylene based material; and
a peel seal attaching the lower vapor barrier layer to the upper vapor barrier layer, thereby forming a fluid tight cavity containing the fluid reservoir,
wherein upon separation of the upper vapor barrier layer and the lower vapor barrier layer the fluid reservoir remains adhered to the lower vapor barrier layer and the fluid reservoir is exposed to the atmosphere and capable of releasing the fragrance.

20. A method of making a device for containing and releasing a volatile substance comprising:
providing a lower vapor barrier layer, having a first surface and a second surface, wherein the second surface comprises a heat sealable thermoplastic material;
disposing a pressure sensitive adhesive on the first surface of the lower vapor barrier layer;
forming a fluid reservoir comprising a composite material comprising a fibrous component and a heat sealable thermoplastic component;
applying a heat seal between the fluid reservoir and the second surface of the lower vapor barrier layer to form a mechanical thermoplastic melt bond between the fluid reservoir and the second surface;
absorbing a volatile substance into the fluid reservoir;
disposing an upper vapor barrier layer directly on the fluid reservoir; and
applying a peel seal between the lower vapor barrier layer and the upper vapor barrier layer, thereby forming a fluid tight cavity containing the fluid reservoir,
wherein upon separation of the upper vapor barrier layer and the lower vapor barrier layer the fluid reservoir remains adhered to the lower vapor barrier layer and the fluid reservoir is exposed to the atmosphere and capable of releasing the volatile substance.
